(19) United States
(12) Patent Application Publication
(10) Pub. No.: US 2015/0289755 A1
(43) Pub. Date: Oct. 15, 2015

(54) MULTI-VIEW IMAGING SYSTEM FOR LAPAROSCOPIC SURGERY

(71) Applicants: CENTRE NATIONAL DE LA RECHERCHE SCIENTIFIQUE (CNRS), Paris (FR); UNIVERSITE JOSEPH FOURIER - GRENOBLE 1, St Martin D'heres (FR); CENTRE HOSPITALIER UNIVERSITAIRE GRENOBLE, La Tronche (FR)

(72) Inventors: Sandrine VOROS, Grenoble (FR); Philippe CINQUIN, St Nazaire Les Eymes (FR); Céline FOUARD, Grenoble (FR); Brahim TAMADAZTE, Besancon (FR)

(73) Assignees: CENTRE NATIONAL DE LA RECHERCHE SCIENTIFIQUE (CNRS), Paris (FR); UNIVERSITE JOSEPH FOURIER - GRENOBLE 1, St Martin D'heres (FR); CENTRE HOSPITALIER UNIVERSITAIRE GRENOBLE, La Tronche (FR)

(21) Appl. No.: 14/433,487

(22) PCT Filed: Oct. 4, 2013
(86) PCT No.: PCT/EP2013/070740

§ 371 Date: Apr. 3, 2015

(30) Foreign Application Priority Data
Oct. 5, 2012 (FR) 1259489

(51) Int. Cl. A61B 1/012; A61B 1/313; A61B 1/05

(52) U.S. Cl. CPC A61B 1/0125; A61B 1/05; A61B 1/3132

(57) ABSTRACT

The invention concerns a multi-view imaging system for laparoscopic surgery comprising: A tubular member; A first imaging device having a longitudinal body and an active end for acquiring images, intended to be inserted through the tubular member, and movable in the tubular member in translation along the longitudinal axis and/or in rotation about the longitudinal axis; A second imaging device comprising at least two cameras each mounted on a support member and movable relative to the tubular member between: a stowed position in which the cameras are positioned inside the tubular member, and a deployed position in which the cameras are positioned outside the tubular member at the distal end, the cameras being arranged on either side of the longitudinal axis of the tubular member and being held secure relative to the tubular member by holding means, so as to follow any movement of the tubular member.

### FIELD OF THE INVENTION

The present invention relates to the field of medical imaging, more particularly imaging within the scope of laparoscopic surgery.

### STATE OF THE ART

Laparoscopic surgery, also-called minimal invasive surgery (MIS), notably used for carrying out intra-abdominal or intra-thoracic operations, requires small incisions, generally smaller than 1 cm, as compared with wide incisions required in laparotomy. This approach gives the possibility of reducing blood losses and post-operative pains, of having small hemorrhages, reducing the recovery period while providing better healing.

Laparoscopic surgery belongs to the wider field of endoscopy which uses imaging systems allowing the viewing of the operating field. An endoscope may consist in a set of lenses laid out in a tube and connected to a video camera, or be a digital laparoscope in which a CCD (Charge-Coupled Device) sensor is positioned at the active end of the laparoscope intended to acquire the images.

In laparoscopic techniques, the surgeons often use two or four specific instruments introduced into the body of the patient through as many trocars. The surgeon bears an instrument in each of his/her hands, so that the endoscope should generally be held by an assistant. Consequently, the surgeon has not total freedom for controlling the viewing of the endoscope.

Robots have therefore been developed for allowing the surgeon to himself/herself control, by voice control for example, the orientation and the zoom of the endoscope, this in a specific way, according to his/her needs during an operation without having to communicate with an assistant. For example the endoscope-holder robot ViKY® marketed by Endocontrol, or further the medical robot daVinci® marketed by Intuitive Surgical may for example be mentioned. Even if these robots have allowed a great improvement in the practice of surgeons, there remain points to be improved, notably as regards the limitation of the field of view provided by these devices, its quality as well as access to the hidden areas in the operating field.

Among the developments of viewing systems for laparoscopic surgery, stereoscopic vision devices have been proposed. For example, document U.S. Pat. No. 5,305,121 proposes replacement of the traditional mono-endoscope with a stereo-endoscope comprising a tube, inside which an illumination based on optical fibers may slide and an arrangement of two CCD cameras mounted on the illumination. Initially, the cameras are pre-positioned in the tube which also contains the light source. Once this tube is introduced into the inside of the abdominal cavity through a trocar, the practitioner pushes the cameras outside the tube via the illumination. Next, the cameras may be oriented relatively to each other by means of SMA (“Shape-Memory Alloy”) actuators. Such a device is intended to provide the practitioner with more complete information on the operating field by means of stereoscopic vision. This stereoscopic vision device, however, comprises the same limits as a mono-endoscope, notably in terms of resolution, depth of field and field of vision. Further, the proposed arrangement imposes a deployment of cameras in the operating field which is not controlled and which may collide with certain internal organs and compromise the surgical operation.

Document U.S. Pat. No. 6,614,595 proposes an imaging system which gives the possibility of widening the customary field of vision, since this document proposes an endoscope combining two optics laid out for providing a stereoscopic view with third optics allowing a wider view. More specifically, integration into a same body of a set of lenses in series is proposed, giving the possibility of bringing the image of the distal portion of the endoscope to the external portion on which a camera is attached. The proposed single-block architecture where the overall viewing system is totally dependent on the stereoscopic viewing system, in terms of orientation and displacement along the optical axis of the endoscope (forward zoom and backward zoom) is shown as giving the possibility of guaranteeing a wide overall field of vision while having sufficient image quality for specific viewing for remote cameras allowing stereoscopic viewing. The proposed architecture is, however, highly complex, and very limited by the size of the body used. Such a system is moreover not optimum for guaranteeing the practitioner an overall view when he/she desires an enlargement with stereoscopic viewing.

Document US 2012/0065468 also proposes an imaging system of the endoscope type, adapted for colonoscopy notably, which provides a widened field of vision as compared with traditional endoscopes. Indeed, provision is made for an endoscope having a cylindrical body with at its distal end, conventionally, a first viewing element, such as a camera, the endoscope further comprising one or several secondary viewing elements attached on the side walls of the cylindrical body, in order to provide a side view in addition to the central view. These side viewing elements give additional information to the practitioner upon advancing the endoscope into the colon, and optionally allows viewing of the specific elements to be treated on the internal walls of said colon. Such a system is, however, not provided for laparoscopic surgeries where it does not have any benefit since the side viewing is not useful for the operation as such. Further, such a device does not give the possibility of providing the practitioner with an overall view of the operating field combined with a specific view of an element selected within this operating field.

Document US 2011/0306832 proposes an endoscope allowing viewing of the operating field according to different view points, or three dimensional viewing of said operating field. For this purpose, an endoscope is proposed, having a distal end at which are attached three deployable arms, each arm bearing a video sensor for allowing viewing of the operating field during the operation. This endoscope may further optionally comprise a camera at its central axis, this central camera being only used for facilitating the insertion of the endoscope as far as the operating field, and not used during the operation as such. Such a viewing system, however, has a complex and highly specific design. Further, it does not give the possibility of providing the practitioner with an overall view of the operating field combined with specific viewing of an element selected within this operating field.

An object of the present invention is therefore to propose an imaging system for laparoscopic surgery which gives the possibility of solving at least one of the aforementioned drawbacks.

In particular, an object of the present invention is to propose an imaging system for laparoscopic surgery which provides the practitioner with a widened field of view while allowing him/her to perform enlargements on a specific area of the operating field.

Still an object of the present invention is to propose a multi-vision imaging system for laparoscopic surgery, simple to use and providing increased safety towards the patient.

Another object of the present invention is to propose a multi-vision imaging system for laparoscopic surgery providing an overall view of the abdominal cavity. This gives the possibility of viewing in a wider way, for example, the introduction of instruments into the abdominal cavity.

Still another object of the present invention is to propose an imaging system for laparoscopic surgery, which may be adapted to existing standard endoscopes.

### DISCUSSION OF THE INVENTION

For this purpose, a multi-vision imaging system for laparoscopic surgery is proposed, characterized in that it comprises:
a tubular member, said tubular member having a longitudinal axis, a distal end and a proximal end;
a first imaging device having a longitudinal body and an active end for image acquisition, the first imaging device being intended to be inserted through the tubular member with the active end protruding with respect to the distal end, and the first imaging device being movable in the tubular member in translation along the longitudinal axis and/or in a rotation around the longitudinal axis;
a second imaging device comprising at least two cameras, each mounted on a supporting member and being displaceable with respect to the tubular member between:
   a retracted position in which the cameras are positioned inside the tubular member, and
   a deployed position in which the cameras are positioned outside the tubular member at the distal end, the cameras being laid out on either side of the longitudinal axis of the tubular member and being held fixed with respect to the tubular member by holding means, so as to follow any movement of the tubular member.

Preferred but non-limiting aspects of this imaging system, taken alone or as a combination, are the following:
the holding means are at least partly formed by the wall of the supporting members intended to bear upon the longitudinal body of the first imaging device in the deployed position.
the holding means comprise return members for maintaining the cameras bearing against the tubular member, in the deployed position.
in the retracted position, the cameras are aligned inside the tubular member along the longitudinal axis.
in the retracted position, the cameras are positioned inside the tubular member facing each other on either side of the longitudinal axis.
in the deployed position, the cameras are positioned so as to have an optical axis parallel to the longitudinal axis.
in the deployed position, the cameras are positioned so that the optical axes of the cameras form an angle comprised between 6° and 15°.
in the deployed position, the cameras are positioned so that the optical axes of the cameras are separated by a distance comprised between 10 millimeters and 30 millimeters.
in the deployed position, the cameras are positioned so that the optical axes of the cameras and the longitudinal axis are in a same plane.
the supporting members are rotatably mounted with respect to the tubular member, the supporting members comprising an actuation lug allowing the longitudinal body to move the supporting members in rotation with view to their deployment upon inserting the first imaging device into the tubular member.
the supporting members are translationally mounted with respect to the tubular member, the supporting members comprising an actuation lug allowing the longitudinal body to translationally move the supporting members with view to their deployment upon inserting the first imaging device into the tubular member.
the tubular member comprises two rails and the supporting members of the cameras comprise a guiding lug intended to cooperate with the rails for guided translation of the supporting members of the cameras with respect to the tubular member.
the tubular member is a trocar intended to be positioned through an incision made in the body of a patient.
the tubular member is an internal adapter intended to be inserted inside a tubular portion of a trocar, the internal diameter of the tubular member being substantially the same as the external diameter of the tubular portion of the trocar.
the tubular member is an external adapter inside which a tubular portion of a trocar is intended to be inserted, the external diameter of the tubular member being substantially the same as the internal diameter of the tubular portion of the trocar.
the first imaging device is an endoscope.

### DESCRIPTION OF THE FIGURES

Other features and advantages of the invention will further become apparent from the description which follows, which is purely illustrative and non-limiting and should be read with reference to the appended drawings, wherein:

FIG. 1 is an illustration of an operating field with surgical tools and the multi-vision imaging system according to the invention;

FIG. 2 is a perspective illustration of the multi-vision imaging system according to the invention;

FIGS. 3A, 3B and 3C illustrate the structure and the operation of the multi-vision imaging system according to a first embodiment of the invention;

FIGS. 4A, 4B and 4C illustrate the structure and the operation of the multi-vision imaging system according to a second embodiment of the invention;

FIGS. 5 and 6 illustrate the structure of the multi-vision imaging system according to a third embodiment of the invention;

FIGS. 7 and 8 illustrate the structure of the multi-vision imaging system according to a fourth embodiment of the invention;

FIG. 9 illustrates a sequence of images taken by a traditional endoscope upon reproducing a suture task in laparoscopy;

FIG. 10 illustrates a sequence of images taken by the multi-vision imaging system according to the invention upon reproducing a suture task in laparoscopy.

### DETAILED DESCRIPTION OF THE INVENTION

The proposed imaging system consists of combining a first imaging device conventionally used by surgeons during laparoscopic operations, for example endoscopy, with a second imaging device providing an overall view of the operating field in which the surgeon intervenes. This second imaging device has the purpose of providing the surgeon with additional information relating to the operative environment, for facilitating his/her intervention without having to necessarily intervene on the first imaging device, notably for moving it. The surgeon for example does without the traditional displacements (introduction and extraction) of the endoscope in order to perform a forward or backward zoom with respect to the operating field. The novel imaging system guarantees an overall view of the operative site while the endoscope ensures a more local and specific view of this site.

In the remainder of the description, the first imaging device described is an endoscope 20 which the surgeon may insert into the operating field through a trocar 10 positioned through an incision made in the patient. However, any imaging device having similar characteristics to endoscopes, notably in terms of shape, may be used. In particular, the first imaging device may operate on infra-red or fluorescence or echographic imaging technologies.

The endoscope 20 has a longitudinal body 21 and an active end 22 for acquiring images. This active end 22 corresponds to the portion of the endoscope 20 intended to be in the operating field for observation. Traditional endoscopes use a set of optical lenses positioned in the longitudinal body 21 from the active end 21 to as far as the opposite end where a camera 40 is generally positioned, connected to a display system, such as a screen, for viewing by the surgeon. With miniaturization of electronics, endoscopes 20, so-called electronic endoscopes, exist today, for which the sensor of the camera is directly positioned at the active end which gives the possibility of avoiding having a complex lens system.

During an operation, the endoscope 20 is inserted through a trocar 10 which ensures the seal between the incision made on the patient and the endoscope. The trocar also ensures the role of an intermediate for introducing CO₂ gas into the abdominal cavity in order to generate a working space for the surgeon. Further, the trocar 10 allows guidance of the movements of the endoscope 20 with respect to the patient. It may be considered that the trocar is a connection of the ball joint type with respect to the body of the patient at the seal element 11 positioned at the incision made in the body of the patient. The endoscope 20 has dimensions adapted so that the longitudinal body 21 may be inserted through the trocar 10, while being movable in the trocar 10 according to a translation along the longitudinal axis L and/or according to a rotation around the longitudinal axis L. Thus, by translating the endoscope 20 with respect to the trocar 10, the practitioner may perform a forward zoom (corresponding to an enlargement), since the active end is brought closer to the area to be observed. Rotation around the longitudinal axis L allows a specific rotation of the endoscope 20 for changing the orientation of the image. Finally, the endoscope 20 may be positioned according to any angle of observation via the ball-joint connection formed at the incision (insertion point of the trocar 10).

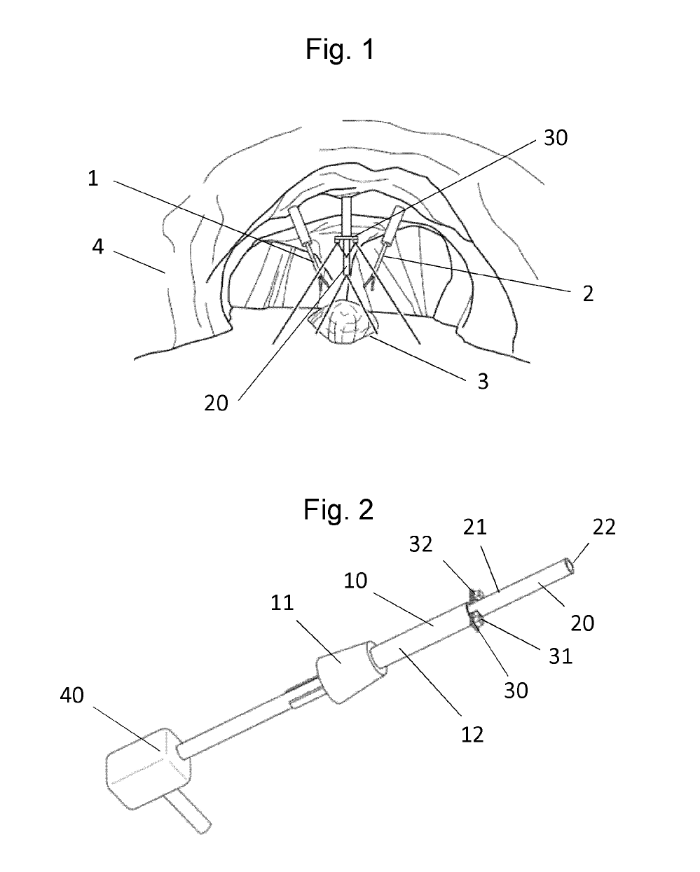

The proposed imaging system has the particularity of having a second imaging device 30 which is provided for giving the surgeon a view of the operating field other than the one provided by the endoscope 20. More particularly, the second imaging device 30 is provided for providing an overall view of the operating field while the first imaging device 20 provides a more localized view, depending on the needs of the surgeon (the endoscope being free to move along and around its optical axis with respect to the second imaging device), of an area of the operating field. With a suitable device for displaying the images, the practitioner may therefore both have a general overview of the operating field while remaining zoomed on the intervention area for example. This is very useful for example when the surgeon has to introduce tools (1, 2) into the operating field 3 or to remove them from the operating field 3, since he/she does not need to move the endoscope in order to see the displacement of these surgical tools in the operating field (see FIG. 1). Further, untimely displacements of the endoscope risk making the optics dirty for example, which would require regular extraction of the endoscope from the abdominal cavity 4, cleaning it, and then re-inserting it into the patient, causing additional operative times. This also allows the surgeon to be able to very accurately estimate the relative position of the surgical tools (1, 2) used, without modifying the position or the orientation of the imaging system.

The second imaging device 30 preferably comprises at least two cameras (31; 32) which are displaceable with respect to the trocar 10 or to another tubular member, forming an adapter, intended to be coupled to the trocar 10 at the tubular portion present in the operating field. More specifically, the cameras (31; 32) are displaceable between a retracted position provided for inserting and/or withdrawing the imaging system in the operating field, and a deployed position during the intervention giving the possibility of having the different views mentioned above. Still preferably, both cameras (31; 32) are movably mounted on the trocar 10 or tubular member, bound rigidly. These cameras (31; 32) remain, however, free with respect to displacements of the endoscope along its optical axis (forward zoom and backward zoom). More specifically, the cameras (31; 32) follow the displacement in rotation of the traditional endoscope (rotation with respect to the insertion point) but they remain fixed when the matter is to displace the endoscope in order to perform a forward or backward zoom. Thus, the association of both imaging devices gives the possibility of both guaranteeing a localized view with the endoscope when it is driven in and a more generalized view (in every case) by means of the second imaging device 30. In the retracted position, the cameras are provided so as to be positioned inside a tubular member 12, i.e. the trocar or the tubular member forming an adapter. Thus, when this tubular member 12 is inserted into the operating field, there is no or little risk of coming into contact undesirably with organs present in the operating field. In the same way, in the retracted position, the removal of the tubular member 12 from the operating field is not hampered by the cameras (31; 32).

In the deployed position, the cameras (31; 32) are positioned outside the tubular member 12 at the distal end of this tubular member 12, i.e. the end which is the closest to the operating field. The cameras (31; 32) are further laid out on either side of the longitudinal axis L of the tubular member 12. Preferably, the camera 31 is positioned symmetrically with respect to the other camera 32 relatively to the longitudinal axis L.

Moreover, in the deployed position, the cameras (31; 32) are held fixed with respect to the tubular member 12 so as to be secured to the tubular member 12, notably in order to have identical displacements with those of the tubular member 12. Suitable holding means give the possibility of securing the cameras (31; 32) to the tubular member 12 in the deployed position, while allowing a displacement of the cameras (31; 32) from or towards the retracted position. As this will be seen later on, these holding means may notably be partly formed by the supporting members 33 on which are mounted the cameras (31; 32), for example in cooperation with the endoscope 20.

The coupling of the cameras (31; 32) to the tubular member 12—which may for example correspond to the trocar 10—gives the possibility of having an overall field of view which is oriented according to the orientation of the trocar around the ball-joint connection, which also corresponds to the orientation of the endoscope 20 in translation in the tubular member 12. However, since the cameras (31; 32) are not coupled with the endoscope 20, this gives the possibility of making certain displacements of the endoscope 20 independent with respect to the second imaging device 30, i.e. translation along the longitudinal axis L of the tubular member 12 and rotation around this same longitudinal axis L. Very advantageously, the endoscope 20 may therefore be brought closer to the intervention area, in order to perform a zoom on a particular organ for example, while retaining an unchanged overall view of the operating field.

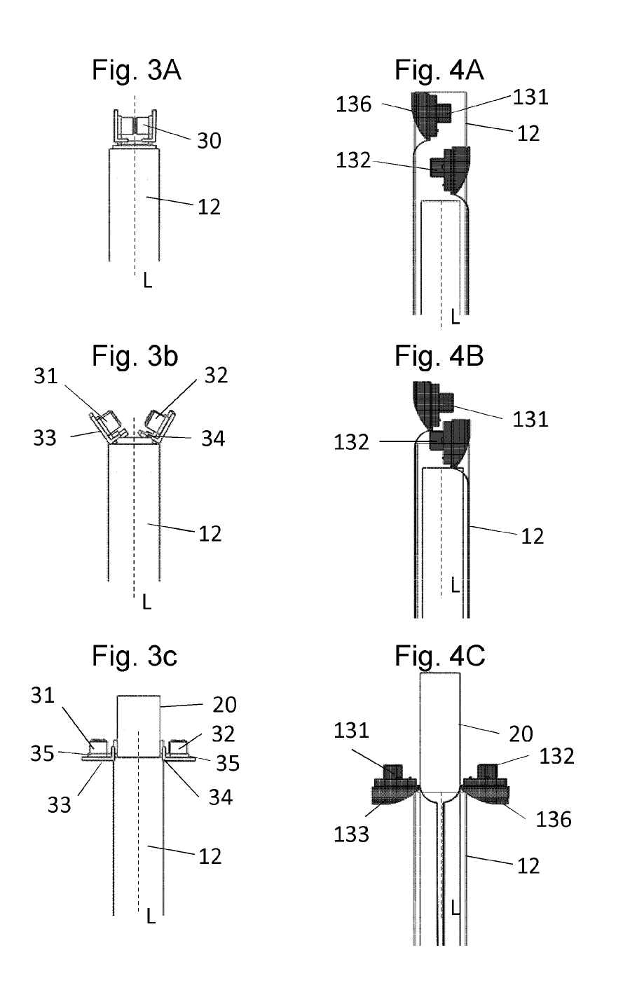

As illustrated in FIG. 2, the cameras (31; 32) of the second imaging device are preferably positioned in the deployed position, on either side of the tubular member 12, and therefore of the endoscope 20. The cameras (31; 32) in this case have a layout “like spectacles” around the endoscope 20. This layout is again found “like spectacles” illustrated in FIGS. 3C and 4C.

The use of at least two cameras (31; 32) on either side of the tubular member 12 has many advantages. As illustrated in FIG. 1, this specific arrangement of the cameras (31; 32) for example gives the possibility of seeing the tip of the endoscope 20, the lateral trocars (trocars for the instruments) as well as the instruments as soon as their introduction into the abdomen of the patient. This gives the possibility of avoiding unpleasant surprises, often encountered by the practitioner upon introducing different tools required for laparoscopic surgery, and considerably reduces the risk of accidents, for example an accidental perforation of a healthy organ.

Moreover, the use of cameras laid out “like spectacles” is very natural for the surgeon, since no realignment among the different images is necessary, which accelerates the learning phase of the practitioner upon using this system. Indeed, a display device with a first screen displaying the image of a first camera 31, a second screen—beside the first screen—displaying the image of the endoscope 20, and a third screen—beside the second screen—displaying the image of the second camera 32 may be contemplated. According to another embodiment, the display system may be coupled with an image processing unit provided for realigning and merging the images of the cameras (31; 32) (a method called mosaicing in the field of computer-aided vision) in order to generate a widened field of view around the image of the endoscope 20.

In the deployed position, the cameras (31; 32) are therefore preferably fixed relatively to the tubular member 12 so that their optical axes are parallel to the longitudinal axis L of the tubular member 12, also corresponding to the optical axis of the endoscope 20. Moreover, the optical axes of the cameras (31; 32) and the longitudinal axis L of the tubular member 12 are preferably in a same plane.

The use of cameras (31; 32) with a stereoscopic view may also be contemplated, in which case the cameras (31; 32) are laid out so that their optical axes form, in the deployed position, an angle comprised between 6° and 15°. Additionally or alternatively, the angle formed by the optical axes of the cameras, may be recalculated after deployment and fixing by mathematical calibration methods (calibration), in order to allow the sought stereoscopic vision.

A special configuration of these cameras (31; 32), in stereovision, would also allow the use of 3D reconstruction approaches in order to provide the surgeon with a 3D browsing environment if this is desired, while retaining an accurate endoscopic image. The reference system given by the endoscope 20 further allows facilitation of the 3D image reconstruction.

The second imaging device 30 is coupled with the tubular member 12 with both simple and rapid means allowing deployment, fixing and retraction with view to withdrawing the imaging system.

Advantageously, it is the insertion of the endoscope 20 into the tubular member 12, and more particularly when the active end 22 of the endoscope 20 passes the distal end of the tubular member 12, towards the operating field, which actuates the deployment of the cameras (31; 32) towards the outside of the tubular member 12.

Preferentially, the supporting members 33 of the cameras (31; 32) are rotatably mounted with respect to the tubular member 12. Further, the supporting members 33 comprise in this case an actuation lug 34 allowing the longitudinal body 21 of the endoscope 20 to move the supporting members 33 in rotation with view to their deployment upon inserting the endoscope 20 into the tubular member 12.

According to an alternative or additional embodiment, the supporting members 33 are translationally mounted with respect to the tubular member 12. In this case, the supporting members 33 comprise an actuation lug 34 allowing the longitudinal body 21 to translationally move the supporting members 33 with view to their deployment upon inserting the endoscope 20 into the tubular member 12.

Thus, in order to pass from the retracted position to the deployed position, the supporting members 33 have a movement of rotation allowing the cameras (31; 32) to be deployed inside the tubular member 12, outwards. This movement of rotation may moreover be coupled with a translational movement allowing the cameras (31; 32) of the inside of the tubular member 12 to be disengaged before their rotation finalizing their deployment.

As indicated above, the holding means allow—in the deployed position—the fixed cameras (31; 32) to be held with respect to the tubular member 12, are at least partly formed by the wall of the supporting members 33 intended to bear upon the longitudinal body 21 of the endoscope 20.

This cooperation between the longitudinal body 21 of the endoscope 20 and the supporting wall of the supporting members 30 further allow the cameras (31; 32) to be held close to the longitudinal axis L of the tubular member 12. Preferably, in the deployed position, the cameras (31; 32) are thus positioned so that their optical axes are separated by a distance comprised between 10 mm and 30 mm. Still more preferably, the optical axis of each camera is at a distance with respect to the wall of the endoscope of less than 10 mm, for example comprised between 2 mm and 8 mm. In the case when the optical axes of the cameras form an angle between them (notably in a stereoscopic configuration), the distances specified above are measured at the optical sensors of the cameras, these optical sensors generally being located at the same level as the cameras as such with respect to the longitudinal axis L.

Moreover, the holding means preferably comprise return means for maintaining the cameras bearing against the tubular member, in the deployed position. These return members may be cables tensioned by the surgeon once the cameras are positioned on the outside of the tubular member 12. These return members may also appear as return springs for example. According to an alternative, the return members are directly formed by the supply flexes of the cameras (31; 32) which simplifies the arrangement of the imaging system.

FIGS. 3A, 3B and 3C illustrate a first embodiment of the proposed imaging system. According to this embodiment, in the retracted position, the cameras (31; 32) are facing each other on either side of the longitudinal axis L. FIG. 3A illustrates this layout of the cameras (31; 32) when they have just been disengaged from the tubular member 12 with view to their deployment. FIG. 3B as for it illustrates the rotation of the cameras (31; 32) towards the deployed position illustrated in FIG. 3C where the cameras (31; 32) have a layout “like spectacles”.

As this is well illustrated in the figures, it is the translation of the endoscope 20 towards the outside of the tubular member 12 which allows deployment of the cameras (31; 32) by actuating a portion 34 of the supporting members 33 of the cameras (31; 32). This same portion 34 also forms the supporting surface of the supporting member 33 on the endoscope 20 which allows the cameras (31; 32) to be held in a fixed position with respect to the tubular member 12 when the endoscope 20, and therefore the cameras (31; 32), are deployed.

Preferably, an integrated circuit 35 is also provided at each supporting member 33 giving the possibility of transmitting the acquired images at the sensors of the cameras (31; 32) towards the processing unit for display.

Such a configuration wherein the cameras (31; 32) are facing each other, preferably inside the tubular member 12, in the retracted position, is possible if the tubular member 12 has a sufficiently wide diameter, or if the cameras (31; 32) are sufficiently small.

In the case when the internal space of the tubular member 12 is too small relatively to the size of the cameras, a layout according to a second embodiment may then be provided wherein, in the retracted position, the cameras are aligned inside the tubular member 12 along the longitudinal axis L. Such a layout is illustrated in FIGS. 4A, 4B, 4C, which respectively illustrate the retracted position, an intermediate position between the retracted position and the deployed position, and the deployed position.

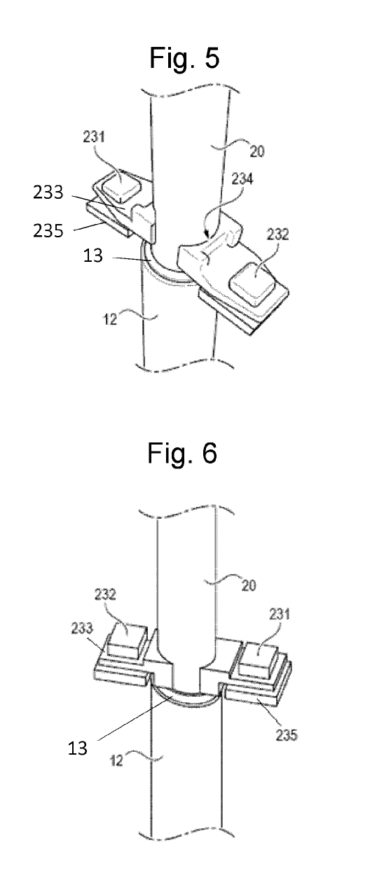

According to still another embodiment illustrated in FIGS. 5 and 6, the cameras (231; 232) are positioned on supporting members 233 free with respect to the tubular member 12. In this case, the supporting members 233 have a very specific shape allowing them to be flattened against the endoscope 20 and against the distal end of the tubular member 12, so that the cameras (231; 232) retain a fixed position relatively to the tubular member 12 when they are deployed. In this respect, provision may further be made for return cables (not shown) which, when they are tensioned, give the possibility of forcing the cameras (231; 232) against the tubular member 12.

Preferably, an integrated circuit 235 is also provided at each supporting member 233 allowing transmission of the acquired images at the sensors of the cameras (231; 232) towards the processing unit for display.

According to this embodiment, in the retracted position, the cameras (231; 232) and associated supporting members 233 are pre-positioned inside the trocar 10, at the distal end. An intermediate tubular member 13 is further positioned inside the trocar 10, this tubular member 13 being used for limiting the play and reinforcing the seal between the trocar 10 and the endoscope 20. The constraining cables as for them run in the intermediate tubular member 13 as far as the outside of the patient, so as to be able to be tensioned from the outside, at the proximal level. More specifically, these cables are positioned between the trocar 10 and the external wall of the intermediate tubular member 13. Not having this intermediate tubular member 13 may also be contemplated.

Once the tubular member 12 is in position in the operating field, the imaging devices may be deployed. To do this, the practitioner pushes the endoscope 20 towards the operating field so that the active end of the endoscope is actually in the operating field. This translation will cause extraction of the cameras (231; 232) from the trocar 10. In order to put them in the deployed position, the constraining cables should then be pulled, which brings the cameras (231; 232) closer to the trocar 10. The shape of the supporting members 233 combined with the stress of the cables will naturally position the cameras (231; 232) bearing both upon the distal end of the trocar 12 and upon the longitudinal body of the endoscope 20.

According to another embodiment of the invention, the cameras are pre-positioned in the tubular member, and are translationally mounted on this tubular member, via slides. For this purpose, the tubular member may comprise rails, appearing as slots for example, and the supporting members of the cameras comprise a guiding lug which may slide in the rail.

The embodiment shown in FIGS. 4A, 4B and 4C integrate such a layout with a system of slides. The guiding lugs 136 cooperate with slots made in the tubular member 12, which may be the trocar or an intermediate tube placed in the trocar.

This slide system has the advantage of facilitating the deployment and the positioning of the cameras. Indeed, the rails cooperating with the shape of the support 133 of the cameras (131; 132) ensuring guidance of the deployment of these cameras (131; 132) as far as their deployed position, preferably in a position symmetrical with respect to the longitudinal axis L of the endoscope 20.

Preferably, an integrated circuit 135 is also provided at each supporting member 133 allowing transmission of the acquired images at the sensors of the cameras (131; 132) towards the processing unit for display.

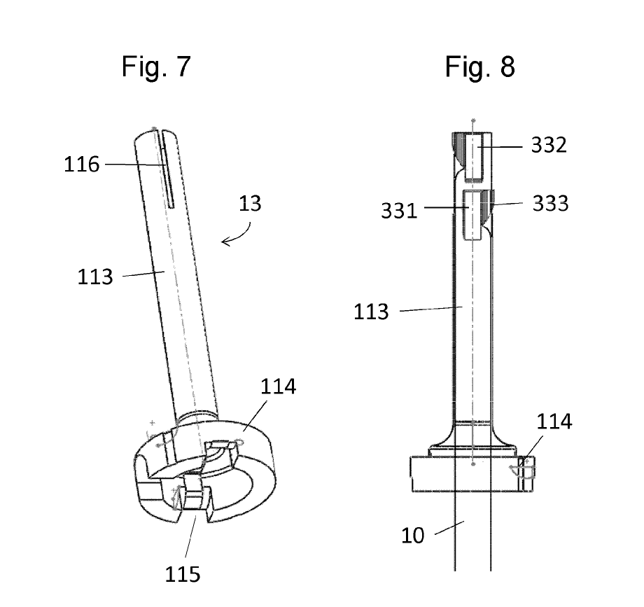

According to still another embodiment as illustrated in FIGS. 7 and 8, a tubular member 13 is provided in which the tubular portion of the trocar 10 is intended to be inserted. This embodiment has the advantage of guaranteeing a total seal of the overall system once it is positioned on the patient.

A trocar generally consists of a tubular portion 12 intended to be inserted into the body of the patient, towards the operating field, and a sealing element 11 positioned at the incision made in the patient. This sealing element 11 notably comprises passages for injecting CO₂ into the operating field or more generally for ensuring the seal of the system.

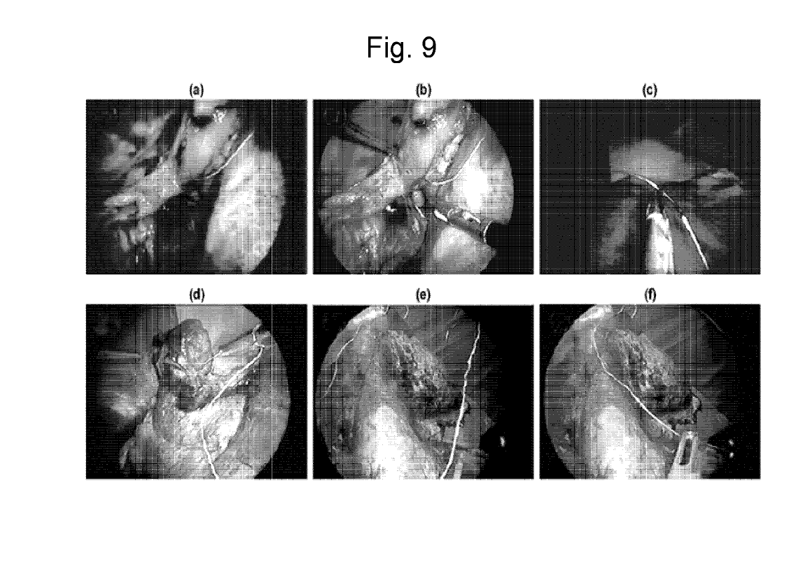

According to the embodiment shown in FIGS. 8 and 9, the tubular member 13 also comprises a tubular portion 113 with a diameter slightly greater than the diameter of the tubular portion 10 of the trocar, and an attachment ring 114 having a shape provided for receiving the sealing element 11 of the trocar. Preferably, the attachment ring 114 comprises apertures 115 provided for receiving the power supplies of the trocar, and/or the cables for maintaining the cameras in position, like the electric power supply flexes of the cameras.

Still preferably, according to this embodiment, the tubular portion 113 of the tubular member 13 comprises a layout with slides as described above, for facilitating the deployment of the cameras on either side of the endoscope. The operation is identical with the operation of the previous embodiments. Indeed, it is the insertion of the endoscope 20 into the tubular portion 10 of the trocar which will push the cameras (331; 332) outside the tubular member 113. The cameras (331; 332) follow a translation along the slots 116 made in the tubular member 113 and being used as a guide for deployment.

In order to withdraw the imaging system from the operating field without accidentally hitting an organ, it is sufficient to withdraw the endoscope 20 and then pull on the cables in order to successively have the first camera 231 and the second camera 232 enter the inside of the tubular member 12, which is itself withdrawn from the incision.

The cameras used for the second imaging device 30 may be varied as long as they meet the problems of required dimensions and resolution. For example, miniature CMOS cameras (5 mm×5 mm×3.8 mm) as proposed by ST Microelectronics may be used, which have the advantage of having a high resolution (1600×1200 pixels), a high frame rate (about 30 frames per second), a low noise to signal ratio, good exposure control (+81 dB), a wide field of vision (51°), as well as a greater field of depth.

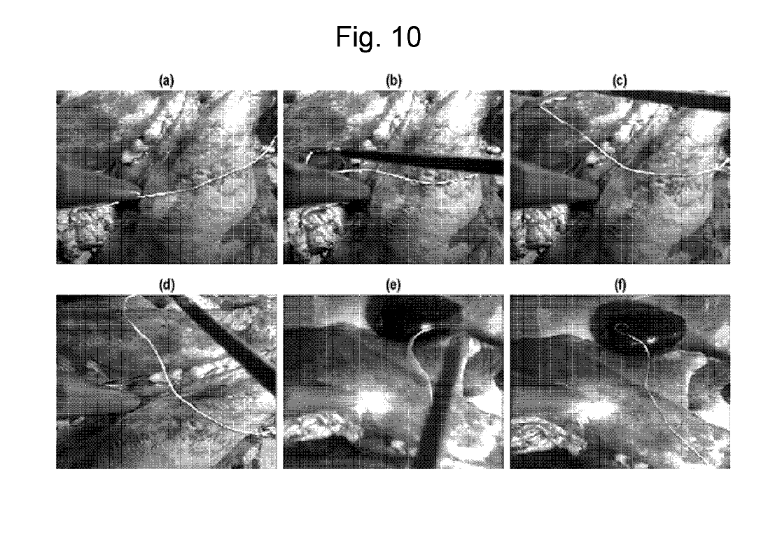

A series of tests was carried out in order to test the effectiveness of the proposed multi-vision imaging system as compared with the use of traditional endoscopes. For this purpose, a test bench was produced, very close to an operation table using pork organs. The investigated scenario consisted of producing a succession of tasks often encountered in the case of laparoscopic surgery, notably of the prostate. In a first case, the surgeon had the task of producing the scenario with the traditional endoscope (cf. FIG. 9) and then reproducing the same tasks with the imaging system integrating global vision (cf. FIG. 10). About 10 cycles were repeated in order to evaluate qualitatively (quality of the suture, comfort for the surgeon/patient, etc.) and quantitatively (required time, number of orders given to the assistant) the contributions of the overall vision system as compared with using a traditional endoscope.

After a first analysis of the results obtained in both scenarios, i.e., producing suture points by means of the traditional endoscope or by using the developed overall vision system, the result was that an average of 3.8 minutes was necessary for successfully completing the task with endoscopic vision and only 29 seconds in the case of the use of the global vision. This represents a gain in time of the order of 10. For a series of tests (5 tasks), 19 minutes are required for the practitioner with the endoscope, while 2.45 minutes are sufficient with the global vision system. It emerges that for the studied procedure, a gain of more than 16 minutes was observed with the system integrating global vision as compared with the traditional endoscope, which is very advantageous for surgical operations.

In addition to the fact that the proposed imaging system is very simple and considerably facilitates the work of the surgeon, thus making him/her more efficient, it also has the advantage of being able to be used with any type of endoscope. Indeed, the configuration of the second imaging device is independent of the endoscope, since it is highly related to the trocar.

Additionally, if the second imaging device may by design be designed in one piece with the trocar, it is possible to use a configuration which may be directly adapted on existing trocars, or connectable via an adaptor.

The reader will have understood that many modifications may be made without materially departing from the new teachings and advantages described here. Therefore, all the modifications of this type are intended to be incorporated within the scope of the presented multi-vision imaging system.

### BIBLIOGRAPHIC REFERENCES

U.S. Pat. No. 5,305,121
U.S. Pat. No. 6,614,595
US 2012/0065468
US 2011/0306832

1. A multi-vision imaging system for laparoscopic surgery comprising:
a tubular member, said tubular member having a longitudinal axis, a distal end and a proximal end;
a first imaging device having a longitudinal body and an active end for acquiring images, the first imaging device being intended to be inserted through the tubular member with the active end protruding with respect to the distal end, and the first imaging device being movable in the tubular member following translation along the longitudinal axis and/or following rotation around the longitudinal axis;
a second imaging device comprising at least two cameras each mounted on a supporting member and being displaceable with respect to the tubular member between:
a retracted position in which the cameras are positioned inside the tubular member, and
a deployed position in which the cameras are positioned outside the tubular member at the distal end, the cameras being laid out on either side of the longitudinal axis of the tubular member and being held fixed with respect to the tubular member by holding means so as to follow any movement of the tubular member.

2. The system according to claim 1, wherein the holding means are at least partly formed by the wall of the supporting members, intended to bear upon the longitudinal body of the first imaging device, in the deployed position.

3. The system according to claim 1, wherein the holding means comprise return members for maintaining the cameras bearing against the tubular member, in the deployed position.

4. The system according to claim 1, wherein, in the retracted position, the cameras are aligned inside the tubular member along the longitudinal axis.

5. The system according to claim 1, wherein, in the retracted position, the cameras are positioned inside the tubular member facing each other on either side of the longitudinal axis.

6. The system according to claim 1, wherein, in the deployed position, the cameras are positioned so as to have an optical axis parallel to the longitudinal axis.

7. The system according to claim 1, wherein, in the deployed position, the cameras are positioned so that the optical axes of the cameras form an angle comprised between 6° and 15°.

8. The system according to claim 1, wherein, in the deployed position, the cameras are positioned so that the optical axes of the cameras are separated by a distance comprised between 10 millimeters and 30 millimeters.

9. The system according to claim 1, wherein, in the deployed position, the cameras are positioned so that the optical axes of the cameras and the longitudinal axis are in a same plane.

10. The system according to claim 1, wherein the supporting members are rotatably mounted with respect to the tubular member, the supporting members comprising an actuation lug allowing the longitudinal body to move the supporting members in rotation with view to their deployment upon inserting the first imaging device into the tubular member.

11. The system according to claim 1, wherein the supporting members are translationally mounted with respect to the tubular member, the supporting members comprising an actuation lug allowing the longitudinal body to move the supporting members in translation with view to their deployment upon inserting the first imaging device into the tubular member.

12. The system according to claim 11, wherein the tubular member comprises two rails and the supporting members of the cameras comprise a guiding lug intended to cooperate with the rails for guided translation of the supporting members of the cameras with respect to the tubular member.

13. The system according to claim 1, wherein the tubular member is a trocar intended to be positioned through an incision made in the body of a patient.

14. The system according to claim 1, wherein the tubular member is an internal adapter intended to be inserted inside a tubular portion of a trocar, the internal diameter of the tubular member being substantially the same as the external diameter of the tubular portion of the trocar.

15. The system according to claim 1, wherein the tubular member is an external adapter inside of which a tubular portion of a trocar is intended to be inserted, the external diameter of the tubular member being substantially the same as the internal diameter of the tubular portion of the trocar.

16. The system according to claim 1, wherein the first imaging device is an endoscope.
